(19) United States
(12) Patent Application Publication
(10) Pub. No.: US 2014/0206023 A1
(43) Pub. Date: Jul. 24, 2014

(54) Methods, Kits & Antibodies for Detecting Intact Fibroblast Growth Factor 21

(71) Applicants: Ping Gao, San Diego, CA (US); Maria Rotoni, San Diego, CA (US)

(72) Inventors: Ping Gao, San Diego, CA (US); Maria Rotoni, San Diego, CA (US)

(21) Appl. No.: 14/164,038

(22) Filed: Jan. 24, 2014

Related U.S. Application Data
(60) Provisional application No. 61/756,390, filed on Jan. 24, 2013.

(51) Int. Cl. G01N 33/566; C07K 16/22

(52) U.S. Cl. CPC G01N 33/566; C07K 16/22
USPC 435/7.92; 530/389.2; 530/388.23; 436/501

(57) ABSTRACT

Disclosed are methods, compositions and kits related to immunoassays for detection of FGF21 using antibodies specific to the N-terminal and/or C-terminal of FGF21. The present invention provides antibodies specific to N-terminal or C-terminal peptide of FGF21. Also provided are immunoassays for specifically measuring intact FGF21 or FGF21 with an untruncated N- or C-terminal region.

### CROSS-REFERENCES AND RELATED APPLICATIONS

This application claims priority to U.S. Provisional Application No. 61/756,390, entitled “Methods, kits & antibodies for detecting intact fibroblast growth factor 21 and the use thereof”, filed Jan. 24, 2013, the content of which is herein incorporated by reference in its entirety.

### BACKGROUND OF THE INVENTION

1. Field of the Invention

The present invention is in the field of immunotechnology. It particularly relates to methods, compositions and kits for measuring intact fibroblast growth factor 21 (FGF21).

2. Description of the Related Art

The human genome encoded for 22 members of the fibroblastic growth factor (FGF) family. Recently, three members of FGF family (FGF19, FGF21 and FGF23) have been identified as metabolic hormones. FGF19 is expressed in intestinal epithelia and regulates biosysthesis of biliary acids and adipogenesis. FGF23 regulates calcium and phosphate homeostasis controlling phosphaturia and vitamin D synthesis. FGF21 is produced mainly in liver. FGF21 is a metabolic regulator with multiple beneficial effects on glucose homeostasis and insulin sensitivity. Some important metabolic effects include (1) increase of glucose uptake in adipocytes, (2) increase of energetic waste and induction of weight loss, (3) reduction of glucose and triglyceride levels in obesity patients and (4) promotion of fatty acid oxidation and increment on ketone bodies formation. Administration of recombinant FGF21 lowered plasma glucose and insulin levels, reduced hepatic and circulating triglycerides and cholesterol levels, and improved insulin sensitivity, energy expenditure, hepatic steatosis and obesity in a range of insulin resistant animal models.

The intact circulating human FGF21 is a small protein comprising 181 amino acids. FGFs share a similar core structure but have significant difference in their N-terminal and C-terminal sequences. The physiological functions of FGF21 are relied on the intact molecular structure and amino acid sequence in its N-terminal and C-terminal region. The N-terminal of FGF21 is biologically active via binding to the FGF receptors (FGFR), while the C-terminal of FGF21 is important and essential for binding to a co-receptor β-Klotho to form a FGF21-β-Klotho-FGFR complex. Yie (Junming Yie, Randy Hecht, Jennifer Patel, et al. FGF21 N- and C-termini play different roles in receptor interaction and activation. FEBS Lett 2009; 583: 19-24.) and Micanovic (Micanovic R, Raches D W, Dunbar J D, et al. Different roles of N and C-termini in the functional activity of FGF21. J Cell Physiol 2009; 219: 227-34.) have reported that the physical interaction of β-klotho with C-terminal segment of FGF21 is necessary for its specific signaling response. Kuro-o (Kuro-o M. Endocrine FGFs and Klothos: emerging concepts. Trends Endocrinol Metab 2008; 19: 239-45.) has reported that when β-klotho is blocked in adipocytes, the effect of FGF21 is inhibited. This indicates the both β-klotho-FGFR complex and the presence of C-terminal FGF21 are essential for FGF21 activity. FGF receptors have an extracellular ligand binding domain formed by three immunoglobulin type domains, a trans-membrane helix and intracellular domain with a tyrosine kinase (TK) activity. The stimulation of specific FGFR by FGF21 leads to the activation of a classical receptor TK signaling pathway with activation of an extracellular response kinase 1/2 (ERK1/2) and Akt (protein kinase B). In beta cells of pancreas, this FGF21-FGFR signaling pathway is usually related with glucose induced activation and nuclear translocation of ERK1/2, which in turn stimulates insulin gene transcription. It is reported that an N-terminal truncated FGF21 (7-181) is a potent inhibitor that competitively inhibits the biological activity of intact FGF21 (1-181) (Junming Yie, et al. FEBS Lett 2009; 583: 19-24).

Circulating FGF21 has been proposed to be a biomarker and its level is increased in patients with nonalcoholic fatty liver disease (NAFLD), type 2 diabetes, gestational diabetes and obesity. An increase of circulating FGF21 is also found in patients with Cushing's syndrome, patients with lipodystrophy induced by HIV-1 and patients with chronic renal disease or end-stage renal disease (ESRD). Measurement of FGF21 concentrations in test specimen such as serum or plasma can be used to identify primary muscle-manifesting respiratory chain deficiencies in adults and children, and might be feasible as a first-line diagnostic test for these disorders, reducing the need for muscle biopsy.

Therefore, it is important to measure the circulating intact FGF21 (1-181) level in order to more accurately assess the biological activity of FGF21 in patients either in the physiological or pathophysiological condition. Current FGF21 immunoassays don't define antibody specificity such as antibody binding epitope. These immunoassays don't describe if the test is measuring intact FGF21 exclusively or if these tests measure N-terminally truncated FGF21 fragment or C-terminally truncated FGF21 fragment. Moreover, the current immunoassays does not describe if the test would also detect other FGF family members. An assay that measures fragments of the FGF21 and/or N-terminally truncated FGF21 and/or C-terminally truncated FGF21 might overestimate the biological activity of the protein in test samples, which may provide misleading information in the clinical diagnosis, monitoring patient condition and treatment, etc. The present technology provides a method for measuring levels of FGF21 with intact N-terminal and/or C-terminal, which can be used to accurately access the biological activities of FGF21 in patient samples.

### SUMMARY OF THE INVENTION

The present invention pertains to immunoassays for measuring levels of FGF21 with intact N-terminal and/or C-terminal amino acid sequences.

One embodiment of the invention provides an isolated antibody and an antigen-binding fragment of the antibody, which can specifically recognize an N-terminal peptide sequence of fibroblast growth factor 21 consisting of HPIPDSSPLLQFGGQVRQ (SEQ ID NO:1), and wherein at least three amino acids in the N-terminal peptide sequence are part of a reactive portion with the antibody or antigen-binding fragment. In a preferred embodiment, the invention provides an isolated antibody and an antigen-binding fragment thereof, which specifically recognize an N-terminal peptide sequence of FGF21 consisting of HPIPDSS (SEQ ID NO:19), wherein at least three amino acids in the N-terminal peptide sequence are part of a reactive portion with the antibody or antibody-binding fragment. The antibody can be a monoclonal or a polyclonal antibody.

Another embodiment of the invention provides an isolated antibody and an antigen-binding fragment of the antibody, which can specifically recognize a C-terminal peptide sequence of FGF21 consisting of DPLSMVGPSQGRSPSYAS (SEQ ID NO:2), and wherein at least three amino acids in said C-terminal peptide sequence are part of a reactive portion with said antibody or antigen-binding fragment. In a preferred embodiment, the invention provides an isolated antibody and an antigen-binding fragment thereof, which specifically recognize an C-terminal peptide sequence of FGF21 consisting of RSPSYAS (SEQ ID NO:20), wherein at least three amino acids in the C-terminal peptide sequence are part of a reactive portion with the antibody or antibody-binding fragment. The antibody can be a monoclonal or a polyclonal antibody.

Another embodiment of the invention provides an immunoassay for measuring the amount of intact FGF21 in a sample. The method comprises the following steps: a) adding to the sample a first antibody or antigen-binding fragment specific for an N-terminal peptide sequence of FGF21 (e.g. an 18-residue peptide), wherein the first antibody or antigen-binding fragment has an epitope with at least three amino acids within the N-terminal peptide sequence of FGF21; b) adding to the sample a second antibody or antigen-binding fragment specific for a C-terminal peptide sequence of FGF21 (e.g. an 18-residue peptide), wherein the second antibody or antigen-binding fragment has an epitope with at least three amino acids within the C-terminal peptide sequence of FGF21; c) allowing the first and second antibody or antigen-binding fragment to bind to intact FGF21 in the sample, thereby forming a complex; d) measuring the amount of the complex to determine the amount of intact FGF21 in the sample while not detecting FGF21 fragments shorter than the intact FGF21. The first and second antibodies can be added simultaneously or sequentially into the sample.

The intact FGF21 used herein, refers to a full-length mature FGF21 protein without the signal peptide. For example, the intact human FGF21 (SEQ ID NO:3) has 181 amino acid residues. The intact FGF21s in other species such as mouse (SEQ ID NO:4), rat (SEQ ID NO:5), monkey (SEQ ID NO:6), cattle (SEQ ID NO:7), and pig (SEQ ID NO:8) have 182, 179, 160, 181, and 181 amino acid residues, respectively. An N-terminal peptide sequence of FGF21 is a peptide located at the N-terminal end of FGF21. The N-terminal peptide of the invention can have at least 7, 8, 9, 10, 11, 12, 13, 14, 15, 16, 17, 18, 19, 20, 21, 22, 23 and 24 amino acid residues. In a preferred embodiment, the N-terminal peptide sequence has 18 amino acid residues. The 18-residue N-terminal peptide sequence can be selected, for example, from the group consisting of SEQ ID NOs: 1, 9, 10, 11, 12 and 13. In another preferred embodiment, the N-terminal peptide has 7 amino acid residues. Similarly, a C-terminal peptide sequence is a peptide located at the C-terminal end of FGF21. The C-terminal peptide of the invention can have at least 7, 8, 9, 10, 11, 12, 13, 14, 15, 16, 17, 18, 19, 20, 21, 22, 23 and 24 amino acid residues. In a preferred embodiment, the C-terminal peptide sequence has 18 amino acid residues. The 18-residue C-terminal peptide sequence can be selected, for example, from the group consisting of SEQ ID NOs: 2, 14, 15, 16, 17 and 18. In another preferred embodiment, the C-terminal peptide has 7 amino acid residues.

One of the first and second antibody can be selected to be a capture antibody, and the other one can be selected to be a detection antibody. The capture antibody is attached to a solid support, allowing a capture antibody-antigen complex to be captured and separated from the solution. The detection antibody is either directly labeled or can react with an labeled compound, allowing a detection antibody-antigen complex to be detected and measured. In this immunoassay of the invention, only intact FGF21 with untruncated N-terminal and untruncated C-terminal can bind to both the first and second antibodies, and therefore be measured.

Another embodiment of the invention provides a method for measuring the amount of FGF21 with an untruncated N-terminal in a sample. The method comprises the following steps: a) adding to the sample an antibody or antigen-binding fragment specific for an N-terminal peptide sequence of FGF21 wherein at least three amino acids in the N-terminal peptide sequence are part of a reactive portion with the antibody or antigen-binding fragment; b) allowing the antibody or antigen-binding fragment to bind to the N-terminal of FGF21 in the sample, thereby forming a complex; and c) measuring the amount of the complex to measure the amount of FGF21 with an untruncated N-terminal in the sample while not detecting FGF21 fragments without an untruncated N-terminal. The N-terminal amino acids of FGF21 are essential for binding to FGF receptor. Measuring the amount of FGF21 with untruncated N-terminal is a way of measuring FGF21 species with FGF receptor binding activity. In a preferred embodiment of the invention, the N-terminal peptide sequence of FGF21 has 18 amino acid residues. In another preferred embodiment of the invention, the N-terminal peptide sequence of FGF21 has 7 amino acid residues.

Another embodiment of the invention provides a method for measuring an amount of FGF21 with an untruncated C-terminal in a sample. The method comprises the following steps: a) adding to the sample an antibody or antigen-binding fragment specific for a C-terminal peptide sequence of FGF21 wherein at least three amino acids in the C-terminal peptide sequence are part of a reactive portion with the antibody or antigen-binding fragment; b) allowing the antibody or antigen-binding fragment to bind to the C-terminal region of FGF21 in the sample, thereby forming a complex; and c) measuring the amount of the complex to measure the amount of FGF21 with an untruncated C-terminal end in the sample while not detecting FGF21 fragments without an untruncated C-terminal end. The C-terminal amino acids of FGF21 are essential for binding to β-klotho. Measuring the amount of FGF21 with untruncated C-terminal is a way of measuring FGF21 species with β-Klotho binding activity. In a preferred embodiment of the invention, the C-terminal peptide sequence of FGF21 has 18 amino acid residues. In another preferred embodiment of the invention, the C-terminal peptide sequence of FGF21 has 7 amino acid residues.

Another embodiment of the invention provides a kit for measuring intact FGF21 in a sample. The kit comprises: a) an antibody or antigen-binding fragment specific for an 18-residue N-terminal peptide sequence of FGF21 wherein at least three amino acids in the 18-residue N-terminal peptide sequence are part of a reactive portion with the antibody or antigen-binding fragment, and b) an antibody or antigen-binding fragment specific for an 18-residue C-terminal peptide sequence of FGF21 wherein at least three amino acids in the 18-residue C-terminal peptide sequence are part of a reactive portion with the antibody or antigen-binding fragment. The kit uses two antibodies each specific for an 18-residue N-terminal or C-terminal peptide of FGF21, respectively, to form a complex with the intact FGF21 in a test sample and specifically measure the amount of the intact FGF21, but not the FGF21 fragments shorter than the intact FGF21.

Another embodiment of the invention provides a method for diagnosing a disease associated with increased or decreased levels of intact FGF21 in a person. The method comprises the following steps: a) obtaining a sample from the person; b) adding to the sample a first antibody or antigen-binding fragment specific for an N-terminal peptide sequence of FGF21 consisting of HPIPDSSPLLQFGGQVRQ (SEQ ID NO:1), wherein at least three amino acids in the N-terminal peptide sequence are part of a reactive portion with the first antibody or antigen-binding fragment; c) adding to the sample a second antibody or antigen-binding fragment specific for a C-terminal peptide sequence of FGF21 consisting of DPLSMVGPSQGRSPSYAS (SEQ ID NO:2), wherein at least three amino acids in the C-terminal peptide sequence are part of a reactive portion with the second antibody or antigen-binding fragment; d) allowing the first and second antibody or antigen-binding fragment to bind to intact FGF21 in the sample, thereby forming a complex; e) measuring the amount of the complex to measure the amount of intact FGF21 while not detecting FGF21 fragments shorter than the intact FGF21; 0 comparing the amount of intact FGF21 in the sample to the range of values from normal people to determine if the person has the disease. One skilled in the field would appreciate that a similar method can also be applied to diagnose a FGF21-related disease in an animal using antibodies specific for N-terminal or C-terminal peptide of FGF21 of the particular animal.

### BRIEF DESCRIPTION OF THE DRAWINGS

FIG. 1. ImmunoAssay Scheme for measuring intact FGF21.

FIG. 2. Condensed ImmunoAssay Protocol.

FIG. 3. ELISA Standard Curve for measuring intact FGF21. The ELISA standard curve was generated by plotting average absorbance at 450 nm/650 nm of duplicate ELISA tests against corresponding concentrations of intact human FGF21.

FIG. 4. Comparison of FGF21 measurements in paired donor EDTA-plasma and serum samples. A) measurement of intact FGF21 in paired donor EDTA-plasma (y-axis) and serum samples x-axis) using the human intact FGF21 ELISA kit of the invention. B) measurement of FGF21 in paired donor EDTA-plasma (y-axis) and serum samples x-axis) using an ELISA kit of other vendor.

FIG. 5. Comparison of FGF21 measurements in normal samples using the invented method x-axis) and other vendor's method (y-axis).

FIG. 6. Comparison of FGF21 measurements in dialysis patients' samples using the invented method x-axis) and other vendor's method (y-axis).

### DETAILED DESCRIPTION

The Physiological function of FGF21 depends on its N-terminal region, which is essential for interacting with FGF receptors and receptor activation, and its C-terminal region, which is essential for binding with β-Klotho proteins. Forming a protein complex of FGF receptor, FGF21 and β-Klotho is critical for FGF21 to exert its biological functions as a metabolism regulator. Therefore, it is important to measure the circulating intact FGF21 (1-181) level in order to more accurately assess the biological activity of FGF21 in patients under either the physiological or pathophysiological condition. Existing technologies for measuring FGF21 do not differentiate among various forms of FGF21. The present invention provides antibodies specific for the N-terminal or C-terminal region of FGF21, allowing measurement of specific FGF21 species with different activities. By specifically measuring FGF21 species with intact N-terminal region, intact C-terminal region or both regions, the present invention provides methods to determine levels of FGF21 species with FGF receptor binding activity, β-Klotho binding activity or both. This allows selection of a particular FGF21 species as a biomarker that can better correlate with a particular FGF21-related diseases. Another advantage of the antibodies of the invention is that these antibodies are less likely to cross-react with other FGF family members. Since all FGF members share a similar core structure but have significant differences in their N-terminal and C-terminal sequences, antibodies specific for the N-terminal or C-terminal region of FGF21 is likely to have higher specificity towards FGF21 over other FGF members.

### GENERAL DEFINITIONS

Unless defined otherwise, all technical and scientific terms used herein have the same meaning as commonly understood by an artisan of ordinary skills in the field to which this invention belongs. The following terms are defined below for the sake of clarity and ease of reference.

The term “FGF21” used herein refers to a mature and circulating form of FGF21 polypeptide in the body, in which the signal peptide at the N-terminal end of the full-length signal peptide-containing FGF21 is removed. The FGF21 of the invention can be FGF21 from any animal species, including but not limited to, human, mouse, rat, dog, cattle and monkey.

The term “intact FGF21” used herein refers to a full-length mature FGF21 polypetide without the signal peptide, wherein the N-terminal region essential for binding to FGF receptors and the C-terminal region essential for binding to β-Klotho are not removed from the FGF21 polypeptide. For example, an intact human FGF21 (SEQ ID NO:3), an intact mouse FGF21 (SEQ ID NO:4), an intact rat FGF21 (SEQ ID NO:5), an intact monkey FGF21 (SEQ ID NO:6), cattle (SEQ ID NO:7), and pig (SEQ ID NO:8) have 181, 182, 179, 160, 181 and 181 amino acid residues, respectively. The intact FGF21 is the biologically active form of the FGF21 with both FGF receptor and β-Klotho binding activities.

The term “FGF21 fragments” used herein refers to a partial polypetide of the intact FGF21 that is shorter than the intact FGF21 polypeptide (e.g. a digested fragment of the intact FGF21), wherein either the N-terminal region responsible for FGF receptor binding and/or the C-terminal region responsible for β-Klotho binding is removed from the polypeptide.

The term “antigen-binding fragment” used herein refers to an antibody fragment that can bind to its antigen. An antigen-binding fragment contains at least one variable domain for binding to antigens. Examples of antigen-binding fragment include the Fab fragment consisting of a variable domain and a constant domain from each of the light and the heavy chain of the antibody, the F(ab′)₂ fragment consisting of two Fab fragments linked by a disulfide bond, the Fd fragment consisting of a variable domain (VH) and part of a constant domain (CH1) of the heavy chain, and the Fv fragment consisting of variable domains of the heavy chain (VH) and the light chain (VL). A single chain Fv fragment where the variable domains of the heavy and the light chain are incorporated into one single polypeptide is also included in the definition.

The term “N-terminal peptide sequence of FGF21” or “N-terminal peptide of FGF21” used herein refers to a peptide sequence located at the N-terminal end of the mature form of FGF21, which is essential for binding to FGF receptor. The N-terminal peptide sequence of FGF21 does not include the signal peptide. For example, an 18-residue N-terminal peptide sequence of FGF21 refers to an 18-amino acid peptide located at the N-terminal end of the mature FGF21. The 18-residue N-terminal peptide sequences of human FGF21, mouse FGF21, rat FGF21, monkey FGF21, cattle FGF21 and pig FGF21 are set forth in SEQ ID NO: 1, SEQ ID NO:9, SEQ ID NO:10, SEQ ID NO:11, SEQ ID NO:12 and SEQ ID NO:13, respectively. Antibodies specific for N-terminal peptide of FGF21 can recognize FGF21 species with FGF receptor binding activity.

The term “C-terminal peptide sequence of FGF21” or “C-terminal peptide of FGF21” used herein refers to a peptide sequence located at the C-terminal end of the mature form of FGF21, which is essential for binding to β-Klotho co-receptor. For example, an 18-residue C-terminal peptide sequence of FGF21 refers to an 18-amino acid peptide located at the C-terminal end of the mature FGF21. The 18-residue C-terminal peptide sequences of human FGF21, mouse FGF21, rat FGF21, monkey FGF21, cattle FGF21 and pig FGF21 are set forth in SEQ ID NO: 2, SEQ ID NO:14, SEQ ID NO:15, SEQ ID NO:16, SEQ ID NO:17 and SEQ ID NO:18, respectively. Antibodies specific for C-terminal peptide of FGF21 can recognize FGF21 species with β-Klotho binding activity.

The term “Capture antibody” used herein refers to an antibody attached or linked to a solid support, which is used to capture the antigen of interest to the solid support and separate it from the solution. The methods to make the capture antibody are well known to those with ordinary skills in the field related to antibodies, and can be found in “Antibodies: a Laboratory Manual” (Eds. Harlow and Lane, Cold Spring Harbor Laboratory Press, NY, 1988). The capture antibody can be directly linked to a solid support. For example, the capture antibody can be chemically linked to agarose beads, magnetic beads, glass beads or plastic beads. The capture antibody can be directly coated to microplates used for immunoassay applications. The capture antibody can also be indirectly attached to a solid support via an interacting partner. For example, a capture antibody can be indirectly attached to a solid support by binding to a secondary antibody which is chemically linked the solid support. A capture antibody or its secondary antibody can be biotinlyated using methods known to people skilled in the art, and the biotinylated antibody can be attached to streptavidin-coated beads or microplates.

The term “Detection antibody” used herein refers to an antibody which is either directly labeled with a detectable marker or can react with a labeled compound (e.g. a fluorescently or enzymatically labeled antibody) to allow the antibody-antigen complex to be detected and measured. The detection antibody can be fluorescently, chemically, enzymatically, or radioactively labeled. For example, to detect a detection antibody by fluorescence, the detection antibody can be chemically linked to a fluorophore such as fluorescein, rhodamine and cyanine, or it can be linked to a fluorescent protein such as green fluorescent protein (GFP) and red fluorescent protein (RFP). The detection antibody can also be detected by colored end products of an enzyme catalyzed reaction. Commonly used enzymes for labeling antibodies include, for example, alkaline phorsphatase and horseradish peroxidase. In some embodiment, the detection antibody is not directly labeled by itself, but is indirectly detected by reacting with a labeled secondary antibody. The methods to incorporate different labels to an antibody are well known to those with ordinary skills in the field, and can be found in the reference book such as “Antibodies: a Laboratory Manual”.

### Antibodies Specific for Recognizing FGF21 N-Terminal and C-Terminal Region.

Members of FGF family share a similar core structure, while there are significant differences in the N-terminal and C-terminal sequences. The present invention provides antibodies that specifically recognize the N-terminal or C-terminal region of FGF21 and are unlikely to cross-react with FGF members other than FGF21. FGF21 has a short half life in the body (e.g. t_{1/2} of human FGF21 is about 30 minutes) and it is quick digested into smaller fragments during the circulation (Ptthoff M J, et al. Gene & Dev, 2012; 26: 312-24). Since N-terminal region of FGF21 is essential for binding to FGF receptor, antibodies specific for the N-terminal region will specifically recognize the FGF21 species with FGF receptor binding activity. Similarly, antibodies specific for the C-terminal region of FGF21 will specifically recognize the FGF21 species with β-Klotho binding activity since the C-terminal region of FGF21 is critical for β-Klotho binding. Using two antibodies specific for N-terminal and C-terminal region of FGF21, the present invention provides a fast and easy method to accurately and specifically measure the level of intact FGF21 with both FGF receptor binding and β-Klotho binding activity in patient's samples.

The FGF21 N-terminal specific antibody of the invention can be developed to specifically recognize an N-terminal peptide of FGF21 with at least 7, 8, 9, 10, 11, 12, 13, 14, 15, 17, 18, 19, 20, 21, 22, 23 and 24 amino acid residues, wherein at least three amino acid residues within the N-terminal peptide are part of the epitope for binding with the antibody. The N-terminal peptide may or may not include the N-terminus amino acid. For example, an N-terminal peptide can be FGF21(1-7), FGF21(1-8), FGF21(1-10), FGF21(1-14), FGF21(1-18), FGF21(2-10), FGFβ-12) and FGF21(2-18). The N-terminal specific antibody of the invention includes any antibody having a part of the epitope located within the N-terminal peptide and another part of the epitope located within the inner region of FGF21. In a preferred embodiment, the FGF21 N-terminal specific antibody can specifically recognize an 18-residue N-terminal peptide of FGF21 selected from the group consisting of SEQ ID NOs:1, 9, 10, 11, 12, and 13. In another preferred embodiment, the FGF21 N-terminal specific antibody recognize a 7-residue N-terminal peptide of FGF21. For example, the anti-N-terminal FGF21 antibody is made by immunizing an animal with N-terminal FGF21(1-7) conjugated to a carrier protein such as Keyhole limpet hemocyanin (KLH) and bovine serum albumin (BSA) and the antiserum is purified by a FGF21(1-7) conjugated resin (see Example 1). The anti-N-terminal FGF21 antibody can also be made by immunizing an animal with N-terminal FGF(1-18) or larger fragment such as FGF21(1-107), and the antiserum is purified by a FGF21(1-7) conjugated resin. It is reported that seven most N-terminal amino acid residues are essential FGF receptor binding. Using antibodies that specifically recognize the seven most N-terminal amino acids can ensure detection of FGF21 with untruncated N-terminal region and therefore FGF receptor binding activity. Antibodies developed against larger fragment of FGF21 can have multiple epitopes and can not specifically target the epitopes at the most N-terminal amino acids. The N-terminal FGF21 specific antibody of the invention allows precise targeting to these biologically active N-terminal residues.

The FGF21 C-terminal specific antibody can be raised against a C-terminal peptide of FGF21 with at least 7, 8, 9, 10, 11, 12, 13, 14, 15, 17, 18, 19, 20, 21, 22, 23 and 24 amino acid residues, wherein at least three amino acid residues within the C-terminal peptide are part of the epitope for binding with the antibody. The C-terminal peptide may or may not include the C-terminus amino acid. For example, a C-terminal peptide of human FGF21 can be FGF21(160-181), FGF21(170-181), FGF21(175-181), FGF21(170-180), FGF21(170-179) and FGF21(165-180). The C-terminal specific antibody of the invention includes any antibody having a part of the epitope located within the C-terminal peptide and another part of the epitope located within the inner region of FGF21. In a preferred embodiment, the FGF21 C-terminal specific antibody can specifically recognize an 18-residue C-terminal peptide of FGF21 selected from the group consisting of SEQ ID NOs:2, 14, 15, 16, 17 and 18. In another preferred embodiment, the FGF21 C-terminal specific antibody recognize a 7-residue C-terminal peptide of FGF21. For example, the anti-C-terminal FGF21 antibody is made by immunizing an animal with C-terminal human FGF21(175-181) conjugated to a carrier protein such as KLH and BSA, and the antiserum is purified by a FGF21(175-181) conjugated resin (see Example 1). The anti-C-terminal FGF21 antibody can also be made by immunizing an animal with C-terminal FGF(164-181) or larger fragment such as FGF21(109-181), and the antiserum is purified by a FGF21(175-181) conjugated resin. It is reported that seven most C-terminal amino acid residues are essential β-Klotho binding. Using antibodies that specifically recognize the seven most C-terminal amino acids can ensure detection of FGF21 with untruncated C-terminal region and therefore β-Klotho binding activity. Antibodies developed against larger fragment of FGF21 can have multiple epitopes and can not specifically target the epitopes at the most C-terminal amino acids. The C-terminal FGF21 specific antibody of the invention allows precise targeting to these biologically active C-terminal residues.

Methods for making antibodies in an animal, screening for antibodies with specificity to FGF21, and purifying the antibodies of interest are well known to those with ordinary skills in the field (see Antibodies: a laboratory manual, 1988). To make antibodies specific for N-terminal or C-terminal region of FGF21, the first step is choose an appropriate immunogen for immunizing an animal. The appropriate immunogen can be a full-length FGF21 protein or a large fragment of FGF21 protein with an intact N-terminal (e.g. human FGF21 (1-107)) or C-terminal sequence (e.g. human FGF21 (109-181)). FGF21 or large fragments will induce generation of antibodies that recognize different epitopes on the protein. Antibodies that specifically recognize N-terminal or C-terminal region of FGF21 can be selected and purified from immunized animal serum using agarose beads or crosslinked polyacrylamide resins immobilized with the N-terminal peptide or C-terminal peptide of FGF21. The appropriate immunogen can also be an N-terminal or C-terminal peptide described above, which is further linked to a carrier protein such as KLH and BSA to increase immunogenicity. An immunologic adjuvant may be used to help stimulate larger immune responses in an animal. Commonly used adjuvants for antibody production include Freund's adjuvant system, Ribi adjuvant system and titermax (Bennett B, et al. J Immunol Methods. 1992; 153(1-2):31-40).

Antibody production involves preparation of immunogen samples and injection of the immunogen into an animal such as a rabbit, a goat, a horse, a rat, a mouse or a chicken to evoke high expression levels of antigen-specific antibodies in the serum, which can be recovered from the animal. Polyclonal antibodies can be directly purified from the animal serum or the egg yoke in the case of chicken. Monoclonal antibodies are produced by fusing antibody-secreting spleen cells from immunized mice with immortal myeloma cells to create monoclonal hybridoma cell lines. The monoclonal hybridoma cell lines expressing antibodies specific for N-terminal or C-terminal region of FGF21 can be selected using the N-terminal or C-terminal peptide described above.

In addition to making antibodies in immunized animals, an antibody specific for the N-terminal or C-terminal region of FGF21 may be obtained from a recombinant antibody library that expresses immunoglobulin variable domains. This method involves four key steps: 1) creation of a recombinant antibody library (e.g. a lambda bacteriophage or a filamentous bacteriophage library); 2) expression of antibody variable domains on the cell surface; 3) screening of antibodies specific for N-terminal or C-terminal region of FGF21; and 4) using affinity maturation to obtain antibodies with high specificity and high affinity (Hoogenboom HR. Nat. Biotechnol. 2005; 23(9):1105-16; WO92/01047). The antibody library may be naive, that is constructed from gene sequences obtained from an organism which has not been immunized with any proteins; or may be constructed using viable domain sequences obtained from an organism which has been exposed to FGF21. The antibody library can then be screened using the N-terminal and C-terminal peptide of FGF21 described above to obtain clones expressing FGF21 antibodies. The obtained antibody-expressing clones can be further optimized using the same FGF21 peptides to get antibodies with high specificity and high affinity for the N-terminal or C-terminal region of FGF21.

### Immunoassays

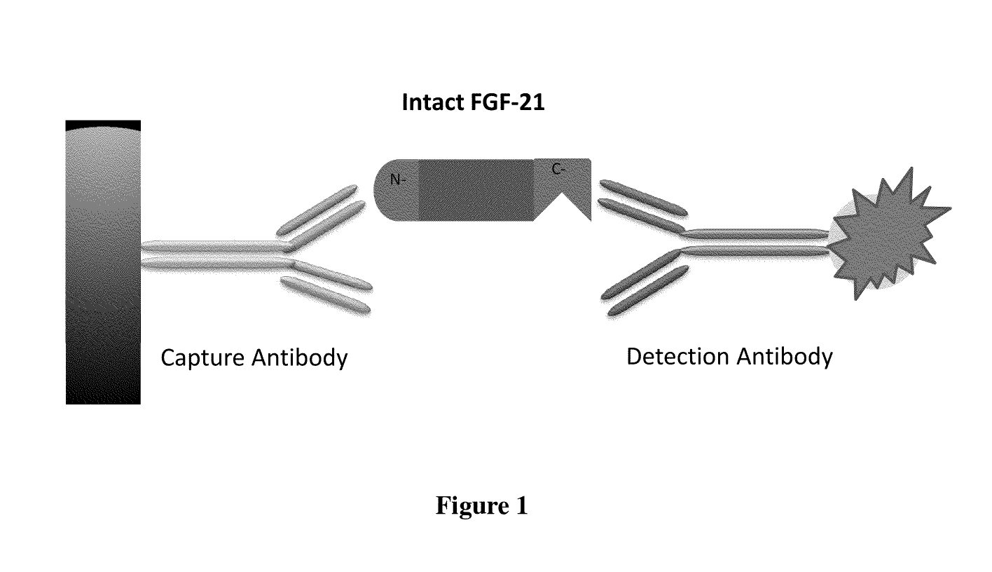

One embodiment of the invention provides an immunoassay (ELISA, enzyme linked immunosorbent assay) for the quantitative measurement of intact FGF21 in a biological sample without detecting FGF21 fragments shorter than intact FGF21. The assay utilizes the two-site “sandwich” technique with two selected antibodies that bind to different epitopes of human intact FGF21 (see FIG. 1). One of the antibodies specifically binds to the untruncated N-terminal amino acid sequence of human FGF21 and the other is specific to the untruncated C-terminal region of human FGF21. One of the two antibodies is selected as a capture antibody and the other one is selected as a detection antibody. Only FGF21 with intact N-terminal and C-terminal region can be first captured by the capture antibody and later detected by the detection antibody. Shorter FGF21 fragments will not be detected in this immunoassay when they lack either the N-terminal or the C-terminal sequence. The two-site “sandwich” immunoassay of the invention comprises the following steps: a) contacting a biological sample with a capture antibody and a detection antibody either simultaneously or sequentially; b) allowing the formation of an antigen-antibody complex containing anti-N terminal FGF21 antibody, intact FGF21, and anti-C terminal FGF21 antibody; c) capturing the antibody-antigen complex and removing unbound antibodies; and d) measuring the amount of the antigen-antibody complex by measuring the detection antibody.

For one embodiment of the invention, an anti-N-terminal human FGF21(1-7) antibody is used as the capture antibody and is coated to wells of immunoassay microplates, and a horseradish peroxidase conjugated anti-C-terminal human FGF21 (175-181) antibody is used as the detection antibody. Assay standards, controls and patient samples are added directly to wells of microplate coated with the capture antibody. Simultaneously, the detection antibody is added to each well. After the first incubation period, the antibody on the wall of microtiter well captures human FGF21 in the sample and unbound proteins in each microtiter well are washed away. A “sandwich” of “capture antibody—human intact FGF21—detection antibody” is formed. The unbound antibody is removed in the subsequent washing step. For the detection of this immunocomplex, the well is incubated with a substrate solution in a timed reaction and measured in a spectrophotometric microplate reader. The enzymatic activity of the immunocomplex bound to human intact FGF21 on the wall of the microtiter well is directly proportional to the amount of intact FGF21 in the sample. A standard curve is generated by plotting the absorbance versus the respective human intact FGF21 concentration for each standard on point-to-point or 4 parameter curve fit. The concentration of human intact FGF21 in test samples is determined directly from this standard curve.

One embodiment of the invention provides an immunoassay method to measure FGF21 with untruncated N-terminal sequence. This method can be used to specifically measure FGF21 species with FGF receptor binding activity. The method comprises the following steps: a) adding to the sample an antibody or antigen-binding fragment specific for an N-terminal peptide of FGF21 wherein at least three amino acids in the N-terminal peptide sequence are part of a reactive portion with the antibody or antigen-binding fragment; b) allowing the antibody or antigen-binding fragment to bind to the N-terminal of FGF21 in the sample, thereby forming a complex; and c) measuring the amount of the complex to measure the amount of FGF21 with an untruncated N-terminal in the sample while not detecting FGF21 fragments without an untruncated N-terminal. The anti-N-terminal FGF21 antibody can be linked to a detectable marker and be directly detected by measuring the detectable marker. It can also be indirectly detected by binding to a labeled secondary antibody which gives detectable signals. In one embodiment of the invention, the proteins in the sample are attached to wells of microplates used for the immunoassay. The anti-N-terminal FGF21 antibody selectively binds to FGF21 with untruncated N-terminal sequence and form an antibody-antigen complex. The unbound antibodies are removed by subsequent washing and the antibody-antigen complex is detected by measuring the detectable markers linked to the antibody. Alternatively, the antibody-antigen complex can be detected by a labeled secondary antibody that recognizes the anti-N-terminal FGF21 antibody.

In another embodiment, the invention provides a two-site “sandwich” immunoassay method for measuring FGF21 with untruncated N-terminal sequence. The method comprises the following steps: a) adding to the sample an antibody or antigen-binding fragment specific for an N-terminal peptide of FGF21 wherein at least three amino acids in the N-terminal peptide sequence are part of a reactive portion with the antibody or antigen-binding fragment, and an antibody that binds to a non-N-terminal region of FGF21; b) allowing the antibody or antigen-binding fragment to bind to the untruncated N-terminal of FGF21 in the sample, thereby forming a complex; and c) measuring the amount of the complex to measure the amount of FGF21 with an untruncated N-terminal in the sample while not detecting FGF21 fragments without an untruncated N-terminal. The anti-N-terminal FGF21 antibody serves as a capture antibody. The measurement is achieved by adding a detection antibody that binds to a non-N-terminal FGF21, i.e. binding to mid-region of FGF21 (90-107), or a detection antibody obtained by immunizing animals with recombinant FGF21 protein as described in the Example 1. The anti-N-terminal FGF21 antibody can be directly coated to a solid phase or indirectly attached to a solid phase via a linker such as biotin that binds to a streptavidin-coated solid phase.

In another embodiment of the invention, an antibody that binds to a non-N-terminal FGF21 region such FGF21 (90-107) is used as a capture antibody, which can be attached to wells of microplates used for the immunoassay. FGF21 proteins and fragments in a test sample are captured to the capture antibody. The anti-N-terminal FGF21 antibody selectively binds to FGF21 with untruncated N-terminal sequence and form an antibody-antigen complex. The unbound antibodies are removed by subsequent washing and the antibody-antigen complex is detected by measuring the detectable markers linked to the anti-N-terminal FGF21 antibody. Alternatively, the antibody-antigen complex can be detected by a labeled secondary antibody that recognizes the anti-N-terminal FGF21 antibody.

In another embodiment, the measurement is achieved by a competitive method known to people skilled in the art. Limited amount of anti-N-terminal FGF21 antibody and known amount of labeled untruncated N-terminal FGF21 are added together to the test sample. The unlabeled untruncated N-terminal FGF21 in the test sample competes with labeled ones to bind to the limited anti-N-terminal FGF21 antibody. The signal generated by the labeled untruncated N-terminal FGF21 is inversely correlated to the amount of untruncated N-terminal FGF21 in the test sample, which can be used to determine the concentration of untruncated N-terminal FGF21 in the test sample.

Another embodiment of the invention provides a method for measuring an amount of FGF21 with an untruncated C-terminal in a sample. This method can be used to specifically measure FGF21 species with β-Klotho binding activity. The method comprises the following steps: a) adding to the sample an antibody or antigen-binding fragment specific for a C-terminal peptide sequence of FGF21 wherein at least three amino acids in the C-terminal peptide sequence are part of a reactive portion with the antibody or antigen-binding fragment; b) allowing the antibody or antigen-binding fragment to bind to the C-terminal of FGF21 in the sample, thereby forming an antibody-antigen complex; and c) measuring the amount of the antibody-antigen complex to measure the amount of FGF21 with an untruncated C-terminal in the sample while not detecting FGF21 fragments without an untruncated C-terminal. In one embodiment, proteins in a sample are coated to wells of microplates for immunoassays, and the antibody specific for C-terminal region of FGF21 is used as a detection antibody to detect FGF21 with untruncated C-terminal. In another embodiment, the anti-C-terminal FGF21 antibody is used as a capture antibody and an antibody that binds to a non-C-terminal FGF21 region such FGF21 (109-181) is used as a detection antibody. In another embodiment, an antibody that binds to a non-C-terminal FGF21 region is used as a capture antibody and the anti-C-terminal antibody is used as a detection antibody. In another preferred embodiment, the amount of FGF21 with untruncated C-terminal region is determined using a competitive method, in which limited amount of anti-C-terminal FGF21 antibody and a known amount of labeled untruncated C-terminal FGF21 are added together to the test sample. The untruncated C-terminal FGF21 in the test sample (unlabeled) competes with the labeled ones to bind to the limited anti-C-terminal FGF21 antibodies. The amount of untruncated C-terminal FGF21 in the test sample is determined by the signal generated by the labeled untruncated C-terminal FGF21, which is inversely correlated to the amount of untruncated C-terminal FGF21 in the test sample.

### Kits for Detecting Different Species of FGF21

Another embodiment of the invention provides a kit for measuring intact FGF21 in a sample. The kit comprises: a) an antibody or antigen-binding fragment specific for an N-terminal peptide sequence of FGF21 (e.g. an 18-residue peptide), wherein at least three amino acids in the N-terminal peptide sequence are part of a reactive portion with the antibody or antigen-binding fragment, and b) an antibody or antigen-binding fragment specific for a C-terminal peptide sequence of FGF21 (e.g. an 18-residue peptide), wherein at least three amino acids in the C-terminal peptide sequence are part of a reactive portion with the antibody or antigen-binding fragment. One of the two antibodies is attached to a solid support (e.g. microbeads and wells of an immunoassay microplate) and used as a capture antibody. The other one is used as a detection antibody. The kit uses an anti-N-terminal FGF21 antibody and an anti-C-terminal FGF21 antibody to form a “sandwich” complex with the intact FGF21 and specifically measure the amount of the intact FGF21, but not the FGF21 fragments shorter than the intact FGF21.

Another embodiment of the invention provides a kit for measuring FGF21 with FGF21 receptor binding activity, comprising an antibody or antigen-binding fragment specific for an N-terminal peptide sequence of FGF21 wherein at least three amino acids in the N-terminal peptide sequence are part of a reactive portion with the antibody or antigen-binding fragment. The kit may further contain a labeled FGF21 with an untruncated N-terminal end.

Another embodiment of the invention provides a kit for measuring FGF21 with b-Klotho binding activity, comprising an antibody or antigen-binding fragment specific for a C-terminal peptide sequence of FGF21 wherein at least three amino acids in the C-terminal peptide sequence are part of a reactive portion with the antibody or antigen-binding fragment. The kit may further contain a labeled FGF21 with an untruncated C-terminal end.

### Clinical Applications

FGF21 plays important roles in glucose and lipid metabolism, and insulin regulation. Change of FGF21 expression was reported in diseases such as primary muscle-manifesting respiratory chain deficiencies, nonalcoholic fatty liver disease and other conditions related to type 2 diabetes, gestational diabetes and obesity. Levels of biologically active FGF21 can be used as a biomarker for aid in diagnosing metabolism-related diseases. An embodiment of the invention provides a method for diagnosing a disease associated with increased or decreased levels of intact FGF21 in a person. The method comprises the following steps: a) obtaining a sample from the person; b) adding to the sample a first antibody or antigen-binding fragment specific for an N-terminal peptide sequence of FGF21 wherein at least three amino acids in the N-terminal peptide sequence are part of a reactive portion with the first antibody or antigen-binding fragment; c) adding to the sample a second antibody or antigen-binding fragment specific for a C-terminal peptide sequence of FGF21 wherein at least three amino acids in the C-terminal peptide sequence are part of a reactive portion with the second antibody or antigen-binding fragment; d) allowing the first and second antibody or antigen-binding fragment to bind to intact FGF21 in the sample, thereby forming a complex; e) measuring the amount of the complex to measure the amount of intact FGF21 while not detecting FGF21 fragments shorter than the intact FGF21; f) comparing the amount of intact FGF21 in the sample to the range of values from normal people to determine if the person has abnormal FGF21 level related to different diseases. In one embodiment of the invention, the N-terminal peptide of FGF21 consists of HPIPDSSPLLQFGGQVRQ (SEQ ID NO:1) and the C-terminal peptide of FGF21 consists of DPLSMVGPSQGRSPSYAS (SEQ ID NO:2). In a preferred embodiment of the invention, the N-terminal peptide of FGF21 consists of HPIPDSS (SEQ ID NO:19) and the C-terminal peptide of FGF21 consists of RSPSYAS (SEQ ID NO:20).

One skilled in the field would appreciate that a similar method can also be applied to diagnose a FGF21-related disease in an animal using antibodies specific for N-terminal or C-terminal peptide of FGF21 of the animal. It is possible that the FGF21 species having FGF receptor binding activity or the ones having β-Klotho binding activity is better correlated with a particular disease. The present invention provides a method to measure levels of N-terminal untruncated FGF21 (having FGF receptor binding activity) or C-terminal untruncated FGF21 (having β-Klotho binding activity), which can be used to diagnose if a person has abnormal level of untruncated N-terminal or C-terminal FGF21 related to particular physiological and pathophysiological status.

Amino acid sequences of FGF21 for some exemplary animals are listed below.

SEQ ID NO: 1
(18-residue N-terminal peptide of human FGF21)
HPIPDSSPLLQFGGQVRQ
SEQ ID NO: 2
(18-residue C-terminal peptide of human FGF21)
DPLSMVGPSQGRSPSYAS
SEQ ID NO: 3
(Human FGF21: 181 aa )
HPIPDS SPLLQFGGQV RQRYLYTDDA QQTEAHLEIR EDGTVGGAAD
QSPESLLQLK ALKPGVIQIL GVKTSRFLCQ RPDGALYGSL
HFDPEACSFR ELLLEDGYNV YQSEAHGLPL HLPGNKSPHR
DPAPRGPARFL PLPGLPPAP PEPPGILAPQ PPDVGSSDPL
SMVGPSQGRS PSYAS
SEQ ID NO: 4
(Mouse FGF21: 182 aa)
AY PIPDSSPLLQ FGGQVRQRYL YTDDDQDTEA HLEIREDGTV
VGAAHRSPES LLELKALKPG VIQILGVKAS RFLCQQPDGA
LYGSPHFDPE ACSFRELLLE DGYNVYQSEA HGLPLRLPQK
DSPNQDATSW GPVRFLPMPG LLHEPQDQAG FLPPEPPDVG
SSDPLSMVEP LQGRSPSYAS
SEQ ID NO: 5
(Rat FGF21: 179 aa)
Y PISDSSPLLQ FGGQVRQRYL YTDDDQDTEA HLEIREDGTV
VGTAHRSPES LLELKALKPG VIQILGVKAS RFLCQQPDGT
LYGSPHFDPE ACSFRELLLK DGYNVYQSEA HGLPLRLPQK
DSQDPATRGP VRFLPMPGLP HEPQEQPGVL PPEPPDVGSS
DPLSMVEPLQ GRSPSYAS
SEQ ID NO: 6
(Rhesus monkey FGF21: 160 aa)
HP IPDSSPLLQF GGQVRQRYLY TDDAQQTEAH LEIREDGTVG
GAAHQSPESE CGPEPGSEGG GALHFDPEAC SFRELLLENG
YNVYQSEAHG LPLHLPGNKS PHRDPASQGP ARFLPLPGLP
PAPPEPPGIL APQPPDVGSS DPLSMVGPSQ ARSPSYAS
SEQ ID NO: 7
(cattle FGF21: 181 aa)
HP IPDSSPLLQF GGQVRQRYLY TDDAQETEAH LEIRADGTVV
GAARQSPESL LELKALKPGV IQILGVKTSR FLCQGPDGKL
YGSLHFDPKA CSFRELLLED GYNVYQSETL GLPLRLPPQR
SSNRDPAPRG PARFLPLPGL PAAPPDPPGI LAPEPPDVGS
SDPLSMVGPS YGRSPSYTS
SEQ ID NO: 8
(pig FGF21: 181 aa)
RPI PDSSPLLQFG GQVRQRYLYT DDAQETEAHL EIRADGTVAG
VARQSPESLL ELKALKPGVI QILGVQTSRF LCQGPDGRLY
GSLHFDPEAC SFRELLLEDG YNVYQSEALG LPLRLPPHRS
SNRDLAPRGP ARFLPLPGLP PAPPEPPGIL APEPPDVGSS
DPLSMVGPSH GRSPSYTS
SEQ ID NO: 9
(18-residue N-terminal peptide of mouse FGF21)
AYPIPDSSPLLQFGGQVR
SEQ ID NO: 10
(18-residue N-terminal peptide of rat FGF21)
YPISDSSPLLQFGGQVRQ
SEQ ID NO: 11
(18-residue N-terminal peptide of monkey FGF21)
HPIPDSSPLLQFGGQVRQ
SEQ ID NO: 12
(18-residue N-terminal peptide of cattle FGF21)
hpipdsspllqfggqvrq
SEQ ID NO: 13
(18-residue N-terminal peptide of pig FGF21)
RPIPDSSPLLQFGGQVRQ
SEQ ID NO: 14
(18-residue C-terminal peptide of mouse FGF21)
DPLSMVEPLQGRSPSYAS
SEQ ID NO: 15
(18-residue C-terminal peptide of rat FGF21)
DPLSMVEPLQGRSPSYAS
SEQ ID NO: 16
(18-residue C-terminal peptide of monkey FGF21)
DPLSMVGPSQARSPSYAS
SEQ ID NO: 17
(18-residue C-terminal peptide of cattle FGF21)
PLSMVGPSYGRSPSYTS
SEQ ID NO: 18
(18-residue C-terminal peptide of pig FGF21)
DPLSMVGPSHGRSPSYTS
SEQ ID NO: 19
(7-residue N-terminal peptide of human FGF21)
HPIPDSS
SEQ ID NO: 20
(7-residue C-terminal peptide of human FGF21)
RSPSYAS
Pongo abelii (Sumatran orangutan): 100% match to
human
HP IPDSSPLLQF GGQVRQRYLY TDDAQQTEAH LEIREDGTVG
GAADQSPESL LQLKALKPGV IQILGVKTSR FLCQRPDGAL
YGSLHFDPEA CSFRELLLED GYNVYQSEAH GLPLHLPGNK
SPHRDPAPRG PARFLPLPGL PPAPPEPPGI LAPQPPDVGS
SDPLSMVGPS QGRSPSYAS
Otolemur garnettii (small-eared galago): 100%
match to human
HPIPDSSPLL QFGGQVRQRY LYTDDAQETE AHLEIREDGT
VVGAAQQSPE SLLELKALKP GVIQILGVKT SRFLCQRPDG
GLYGSLYFDP KACSFRELLL EDGYNVYWSE TYGLPLHLPP
ANSPYWGPSL RSPARFLPLP GPPAASPELP GILALEPPDV
GSSDPLSMVG PSQGRSPSYA S
Canis lupus familiaris (dog): 100% match to human
HP IPDSSPLLQF GGQVRQRYLY TDDAQETEAH LEIRADGTVV
GAARQSPESL LELKALKPGV IQILGVKTSR FLCQGPDGTL
YGSLHFDPVA CSFRELLLED GYNIYHSETL GLPLRLRPHN
SAYRDLAPRG PARFLPLPGL LPAPPEPPGI LAPEPPDVGS
SDPLSMVGPS QGRSPSYAS

### EXAMPLES

### Example 1

### Preparation of Antibodies Specific for N-Terminal Portion and C-Terminal Portion of FGF21

### Production of an Anti-N-Terminal FGF21 Antibody.

An anti-N-terminal FGF21 antibody was prepared using a 7-residue N-terminal peptide FGF21(1-7) (SEQ ID NO:19). Briefly, FGF21(1-7)-Cys was chemically synthesized and conjugated to maleimide-activated KLH ((Pierce, part of Thermo Scientific, Waltham, Mass.)). This conjugated peptide was mixed with Freund adjuvant (using Freund complete adjuvant for primary immunization and Freund incomplete adjuvant for subsequent immunizations) and used to immunize a sheep to produce specific anti-N-terminal FGF21 antiserum.

The anti-FGF21 (1-7) specific antibody was then purified from the antiserum using a column filled with FGF21 (1-7)-(Lys)ₙ-Cys conjugated resin as follows. Firstly, an N-terminal FGF21(1-7)-(Lys)ₙ-Cys peptide was chemically synthesized (n is usually 0 to 5; Lys can also be replaced with other amino acid such as Gly. On the other hand, PEG can also be used to replace the Lys) and conjugated to crosslinked agarose beads using a SulfoLink immobliztion kit (Pierce, part of Thermo Scientific, Waltham, Mass.). Secondly, the anti-N-terminal FGF21 antiserum was filtered via a 0.2 μm pore size membrane, and the anti-N-terminal FGF21 antiserum was diluted by adding the same volume of 0.01M phosphate buffered saline, pH 7.2. Thirdly, the diluted antiserum was loaded to the column and the anti-FGF21(1-7) specific antibody was allowed to bind to FGF(1-7)-(Lys)ₙ-Cys conjugated resin, and unbound proteins were washed away with 0.01M phosphate buffered saline, pH 7.2. The anti-N-terminal FGF21(1-7) antibody was eluted with a 0.1M Glycin-HCl buffer, pH 2.8, and the eluted fractions with detectable amounts of antibody were collected and pooled together. Finally, the purified antibody was neutralized by dialyzing against 0.01M phosphate buffered saline, pH 7.2, with at least three changes of the dialysis buffer.

Anti-N-terminal FGF21 antibodies can be made against other N-terminal peptides of FGF21 using the method described above. For example, a 18-residue N-terminal peptide of FGF21 selected from the group of SEQ ID NO:1, SEQ ID NO:9, SEQ ID NO:10, SEQ ID NO:11, SEQ ID NO:12 and SEQ ID NO:13 can be used to develop an anti-N-terminal FGF21 antibody. A Cys is added to the C-terminus of the 18-residue N-terminal peptide of FGF21, which allows the N-terminal peptide to be conjugated to a maleimide-activated carrier protein such as BSA and KLH. The conjugated N-terminal peptide is then used to immunize an animal such as a sheep, a goat, a rabbit and a chicken to produce a specific anti-N-terminal FGF21 antiserum. The anti-N-terminal FGF21 antibody is further purified from the antiserum using an affinity column filled with an FGF21 N-terminal peptide-conjugated resin. The FGF21 N-terminal peptide used for antibody purification can be the 18-residue N-terminal peptide or shorter peptide such as a 7-residue N-terminal peptide of FGF21.

### Production of Anti-C-Terminal FGF21 Antibody

An anti-C-terminal FGF21 antibody was prepared using a 7-residue C-terminal peptide of human FGF21(175-181) (SEQ ID NO:20). Briefly, Cys-FGF21(175-181) was chemically synthesized and conjugated to maleimide-activated KLH (Pierce, part of Thermo Scientific, Waltham, Mass.). This conjugated peptide was mixed with Freund adjuvant (using Freund complete adjuvant for primary immunization and Freund incomplete adjuvant for subsequent immunizations) and used to immunize a sheep to produce specific anti-C-terminal FGF21 antiserum.

The anti-FGF21 (175-181) specific antibody was then purified from the antiserum using a column filled with Cys-(Lys)ₙ-FGF21(175-181) conjugated resin as follows. Firstly, a C-terminal Cys-(Lys)ₙ-FGF21(175-181) peptide was chemically synthesized (n is usually 0 to 5; Lys can also be replaced with other amino acid such as Gly. On the other hand, PEG can also be used to replace the Lys) and conjugated to crosslinked agarose beads using a SulfoLink immobliztion kit (Pierce, part of Thermo Scientific, Waltham, Mass.). Secondly, the anti-C-terminal FGF21 antiserum was filtered via a 0.2 μm pore size membrane, and the anti-C-terminal FGF21 antiserum was diluted by adding the same volume of 0.01M phosphate buffered saline, pH 7.2. Thirdly, the diluted antiserum was loaded to the column and the anti-FGF21(175-181) specific antibody was allowed to bind to Cys-(Lys)ₙ-FGF21(175-181) conjugated resin, and unbound proteins were washed away with 0.01M phosphate buffered saline, pH 7.2. The anti-C-terminal FGF21(175-181) antibody was eluted with a 0.1M Glycin-HCl buffer, pH 2.8, and the eluted fractions with detectable amounts of antibody were collected and pooled together. Finally, the purified antibody was neutralized by dialyzing against 0.01M phosphate buffered saline, pH 7.2, with at least three changes of the dialysis buffer.

Anti-C-terminal FGF21 antibodies can be made against other C-terminal peptides of FGF21 using the method described above. For example, a 18-residue C-terminal peptide of FGF21 selected from the group of SEQ ID NO:2, SEQ ID NO:14, SEQ ID NO:15, SEQ ID NO:16, SEQ ID NO:17 and SEQ ID NO:18 can be used to develop an anti-C-terminal FGF21 antibody. A Cys is added to the N-terminus of the 18-residue C-terminal peptide of FGF21, which allows the peptide to be conjugated to a maleimide-activated carrier protein such as BSA and KLH. The conjugated C-terminal FGF21 peptide is used to immunize an animal such as a sheep, a goat, a rabbit and a chicken to produce specific anti-C-terminal FGF21 antiserum. The anti-C-terminal FGF21 antibody is further purified from the antiserum using an affinity column filled with an FGF21 C-terminal peptide-conjugated resin. The FGF21 C-terminal peptide used for antibody purification can be the 18-residue C-terminal peptide or a shorter peptide such as a 7-residue C-terminal peptide of FGF21.

### Production of Anti-FGF21 Polyclonal Antibody

Recombinant FGF21 protein or large fragment of FGF21 is expressed in E. coli, CHO or HEK239 cell lines. The purified protein is used to immunize a sheep to produce anti-FGF21 antiserum. Anti-FGF21 antibody is further purified from the antiserum via affinity purification using FGF21-resin column or a Protein A or protein A/G column. This FGF21 antibody can be used in the measurement of untruncated N-terminal FGF21 or untruncated C-terminal FGF21 assay.

### Example 2

### ELISA Assay for Detecting Intact FGF21

The “sandwich” ELISA (enzyme-linked immunosorbent assay) is designed for the quantitative determination of human intact FGF21 level in EDTA-plasma or serum. This assay specifically measures intact FGF21, but does not detect shorter FGF21 fragments. The test is useful as an aid in diagnosis of diseases with increased or decreased levels of FGF21 proteins such as primary muscle-manifesting respiratory chain deficiencies, nonalcoholic fatty liver disease and other conditions related to type 2 diabetes, gestational diabetes and obesity.

### Reagents: Preparation and Storage

This intact human FGF21 ELISA kit must be stored at 2-8° C. upon receipt. Prior to use allow all reagents to come to room temperature. Regents from different kit lot numbers should not be combined or interchanged.

1. Anti-Human FGF21 Antibody Coated Microplate

One microplate with 12×8 well-breakable strips (96 wells total) coated with antibody specific to N-terminal of human FGF21. The plate is framed and sealed in a foil zipper bag with a desiccant. This reagent should be stored at 2-8° C. and is stable until the expiration date on the kit box.

2. Human FGF21 Detection Antibody

One vial containing 0.4 mL concentrated HRP labeled antibody specific to C-terminal of human FGF21 in a stabilized protein matrix. This reagent must be diluted with FGF21 Detection Antibody Diluent before use. This reagent should be stored at 2-8° C. and is stable until the expiration date on the kit box.

3. FGF21 Detection Antibody Diluent

One vial containing 8 mL ready-to-use buffer. It should be only used for detection antibody dilution according to the assay procedures. This reagent should be stored at 2-8° C. and is stable until the expiration date on the kit box.

4. ELISA Wash Concentrate

One bottle containing 30 mL of 30 fold concentrate. Before use the contents must be diluted with 870 mL of demineralized water and mix well. Upon dilution this yields a working wash solution containing a surfactant in phosphate buffered saline with a non-azide, non-mercury preservative. The diluted should be stored at room temperature and is stable until the expiration date on the kit box.

5. ELISA HRP Substrate

One bottle containing 12 mL of tetramethylbenzidine (TMB) with stabilized hydrogen peroxide. This reagent should be stored at 2-8° C. and is stable until the expiration date on the kit box.

6. ELISA Stop Solution

One bottle containing 12 mL of 0.5 M sulfuric acid. This reagent should be stored at 2-8° C. or room temperature and is stable until the expiration date on the kit box.

7. Human FGF21 Standards

Six vials each containing a different concentration of human FGF21 in a lyophilized bovine serum based matrix with a non-azide, non-mercury preservative. Refer to vial for exact concentration for each standard. The standards are ready to use. These reagents should be stored at 2-8° C. and are stable until the expiration date on the kit box.

8. Human FGF21 Controls

Two vials each containing a different concentration of human FGF21 in a lyophilized bovine serum based matrix with a non-azide, non-mercury preservative. Refer to vials for exact concentration range for each control. The controls are ready to use. Both controls should be stored at 2-8° C. and are stable until the expiration date on the kit box.

### Specimen Collection:

Only 50 μL of human EDTA-plasma is required for human FGF21 measurement in singlet. No special preparation of individual is necessary prior to specimen collection. Whole blood should be collected with lavender-top Vaccutainer and separate the plasma from cells by centrifugation (850-1500×g for 10 minutes). The plasma should be separated from the cells right after collection or at least within one hour of blood collection. The plasma should be transferred to a clean test tube right after centrifugation. Plasma samples should be stored at −20° C. if the assay is not to be performed within 48 hours. Avoid more than three times freeze-thaw cycles of specimen.

Serum sample can also be used for FGF21 measurement. Serum sample collection should be performed as suggested by the manufacturer of the sample collection tubes.

### Specimen Shipment:

Collected EDTA-plasma or serum samples should be shipped to designated laboratory in frozen condition with dry ice. If frozen condition is not available, samples should be shipped at room temperature in an insulated container for a maximum of 48 hours. Samples must not be shipped refrigerated, such as with blue ice pack.

### Assay Procedure

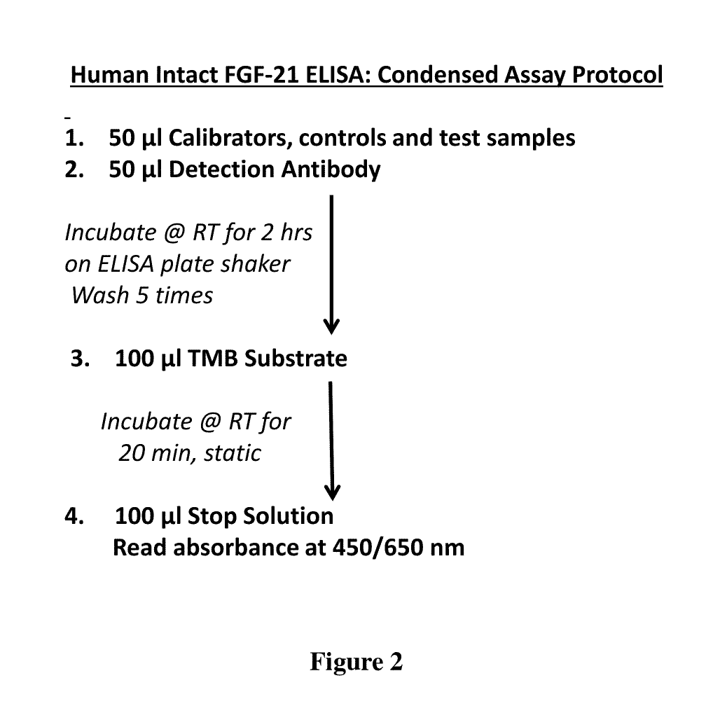

A condensed assay protocol is shown in FIG. 2.

1. Reagent Preparation
(1) Prior to use, allow all reagents to come to room temperature. Regents from different kit lot numbers should not be combined or interchanged.
(2) ELISA Wash Concentrate must be diluted to working solution prior use. Please see REAGENTS section for details.
(3) Reconstitute kit standards and controls by adding 0.5 mL distilled water into each vial. Gently mix and dissolve the entire particle before use. The reconstituted standards and controls should be stored at −20° C. right after use.
(4) Prepare working human FGF21 detection antibody (Cat#30620) by 1:21 fold dilution of the conjugation antibody with the FGF21 Detection Antibody Diluent (Cat#30600). Following is a table that outlines the relationship of strips used and antibody mix prepared.

| | FGF21 | |
|---|---|---|
| | Detection | FGF21 |
| | Antibody | Detection |
| Strip no | Diluent | Antibody |
| 1 | 500 μL | 25 μL |
| 2 | 1000 μL | 50 μL |
| 3 | 1500 μL | 75 μL |
| 4 | 2000 μL | 100 μL |
| 5 | 2500 μL | 125 μL |
| 6 | 3000 μL | 150 μL |
| 7 | 3500 μL | 175 μL |
| 8 | 4000 μL | 200 μL |
| 9 | 4500 μL | 225 μL |
| 10 | 5000 μL | 250 μL |
| 11 | 5500 μL | 275 μL |
| 12 | 6000 μL | 300 μL |

Note:
this antibody mixture must be freshly prepared right before testing.

2. Assay Procedure
(1) Place a sufficient number of antibody coated microwell strips in a holder to run human intact FGF21 standards, controls and unknown samples in duplicate.
(2) Test Configuration

| ROW | STRIP 1 | STRIP 2 | STRIP 3 |
|---|---|---|---|
| A | STD 1 | STD 5 | SAMPLE 1 |
| B | STD 1 | STD 5 | SAMPLE 1 |
| C | STD 2 | STD 6 | SAMPLE 2 |
| D | STD 2 | STD 6 | SAMPLE 2 |
| E | STD 3 | C 1 | SAMPLE 3 |
| F | STD 3 | C 1 | SAMPLE 3 |
| G | STD 4 | C 2 | |
| H | STD 4 | C 2 | |

(3) Add 50 μL of standards, controls and patient plasma/serum samples into the designated microwell.
(4) Add 50 μL of 1:21 diluted detection antibody to each well
(5) Cover the plate with one plate sealer and incubate plate with orbital shaking 170 rpm at room temperature for 2 hours.
(6) Remove plate sealer. Aspirate the contents of each well. Wash each well 5 times by dispensing 350 μL of working wash solution into each well and then completely aspirating the contents. Alternatively, an automated microplate washer can be used.
(7) Add 100 μL of ELISA HRP Substrate into each of the wells.
(8) Cover the plate with one plate sealer and also with aluminum foil to avoid exposure to light. Incubate plate at room temperature for 20 minutes.
(9) Remove the aluminum foil and plate sealer. Add 100 μL of ELISA Stop Solution into each of the wells. Mix gently.
(10) Read the absorbance at 450/650 nm within 10 minutes in a microplate reader

### Procedural Notes

1. It is recommended that all standards, controls and unknown samples be assayed in duplicate. The average absorbance reading of each duplicate should be used for data reduction and the calculation of results.
2. Keep light sensitive reagents in the original amber bottles.
3. Store any unused antibody coated strips in the foil zipper bag with desiccant to protect from moisture.
4. Careful technique and use of properly calibrated pipetting devices are necessary to ensure reproducibility of the test.
5. Incubation times or temperatures other than those stated in this insert may affect the results.
6. Avoid air bubbles in the microwell as this could result in lower binding efficiency and higher CV % of duplicate reading
7. All reagents should be mix gently and thoroughly prior use. Avoid foaming.
8. Since there is no Gold Standard concentration available for human intact FGF21 measurement, the values of assay standards were established by correlation to a highly purified FGF21 standard.
9. For sample values reading greater than highest standard, it is recommend to re-assay samples with dilution.
10. Bacterial or fungal contamination of plasma specimens or reagents, or cross contamination between reagents may cause erroneous results.
11. Water deionized with polyester resins may inactive the horseradish peroxidase enzyme.

### Interpretation of Results

1. Calculate the average absorbance for each pair of duplicate test results.
2. Subtract the average absorbance of the STD 1 (0 ng/mL) from the average absorbance of all other readings to obtain corrected absorbance.
3. The standard curve is generated by the absorbance of all standards. Appropriate computer assisted data reduction programs may also be used for the calculation of results.
   The human intact FGF21 concentrations for the controls and patient samples are read directly from the standard curve using their respective corrected absorbance.

### Example Data and Standard Curve

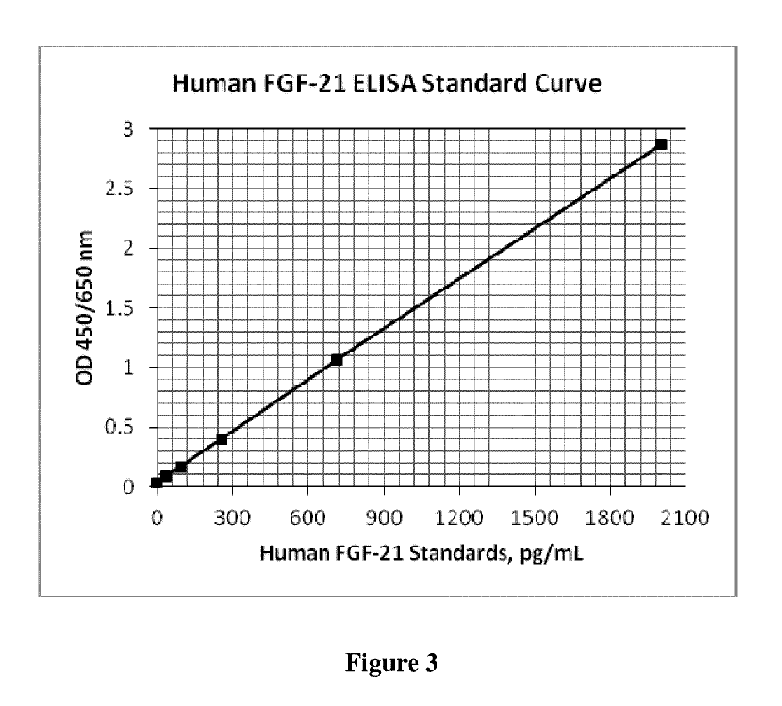

A standard curve for FGF21 ELISA was generated by plotting OD_{450/650} absorbance (corrected) vs. corresponding FGF21 concentration standards (FIG. 3).

| | OD 450/650 nm Absorbance | | | Results |
|---|---|---|---|---|
| | readings | average | corrected | pg/mL |
| 0 | 0.037 | 0.037 | 0.000 | |
| pg/mL | 0.036 | | | |
| 32.5 | 0.087 | 0.087 | 0.050 | |
| pg/mL | 0.086 | | | |
| 91 | 0.172 | 0.170 | 0.133 | |
| pg/mL | 0.169 | | | |
| 255 | 0.398 | 0.399 | 0.302 | |
| pg/mL | 0.399 | | | |
| 714 | 1.067 | 1.068 | 1.031 | |
| pg/mL | 1.069 | | | |
| 2000 | 2.835 | 2.869 | 2.946 | |
| pg/mL | 2.903 | | | |
| Control 1 | 0.126 | 0.127 | 0.371 | 60.83 |
| | 0.129 | | | |
| Control 2 | 0.736 | 0.729 | 1.200 | 481.29 |
| | 0.721 | | | |

### Performance Characteristics

### 1. Sensitivity (LoD)

The sensitivity (lowest detection limit of this human intact FGF21 ELISA) as determined by the corresponding OD value of 2 fold standard deviation above the mean on 20 duplicate determinations of zero standard is 1.7 pg/mL.

### 2. High Dose “Hook” Effect

This assay has showed that it did not have any high dose “hook” effect up to 20,000 pg/mL.

### 3. Precision

The intra-assay precision was validated by measuring three donor EDTA-plasma samples in a single assay with 16-replicate determinations. The results are shown in the table below. CV stands for coefficient of variation from the mean value.

| Mean Human Intact FGF21 | |
|---|---|
| Value (pg/mL) | CV (%) |
| 63.2 | 5.7 |
| 171 | 4.2 |
| 480 | 5.4 |

The inter-assay precision was validated by measuring three control samples in duplicate in 12 individual assays. The results are shown in the table below.

| Mean Human Intact FGF21 | |
|---|---|
| Value (pg/mL) | CV (%) |
| 69.8 | 6.9 |
| 181 | 3.0 |
| 486 | 1.9 |

### 4. Linearity

Two human EDTA-plasma samples were sequentially diluted with 0.01M PBS, pH 7.4 and concentrations of intact FGF21 were measured using the human intact FGF21 ELISA. The results in the value of pg/mL are as follows:

| | | OBSERVED | EXPECTED | RECOVERY |
|---|---|---|---|---|
| # | DILUTION | VALUE | VALUE | % |
| 1 | Neat | 286 | — | — |
| | 1:2 | 138 | 143 | 96 |
| | 1:4 | 75 | 72 | 104 |
| | 1:8 | 37.9 | 36 | 105 |
| | 1:16 | 19.5 | 18 | 108 |
| 2 | Neat | 61.8 | — | — |
| | 1:2 | 32.1 | 30.9 | 104 |
| | 1:4 | 15.9 | 15.5 | 103 |
| | 1:8 | 7.2 | 7.7 | 94 |

### 5. Spike Recovery

Two patient samples were spiked with various amounts of human intact FGF21 (1 vol.+1 vol. mixture) and were assayed for intact FGF21 concentrations. The results in the value of ng/mL are shown in the following table.

| | Orig. | Amount | Observed | Expected | Recovery |
|---|---|---|---|---|---|
| # | Value | Spiked | Value | Value | % |
| 1 | 45.9 | 91 | 64.9 | 68.5 | 95 |
| | (serum) | 255 | 150 | 151 | 100 |
| | | 714 | 388 | 380 | 102 |
| 2 | 40.4 | 91 | 71.2 | 65.7 | 108 |
| | (plasma) | 255 | 148 | 148 | 100 |
| | | 714 | 406 | 377 | 108 |

### Example 3

### Comparison of FGF21 Measurements in Paired Donor EDTA-Plasma and Serum Samples

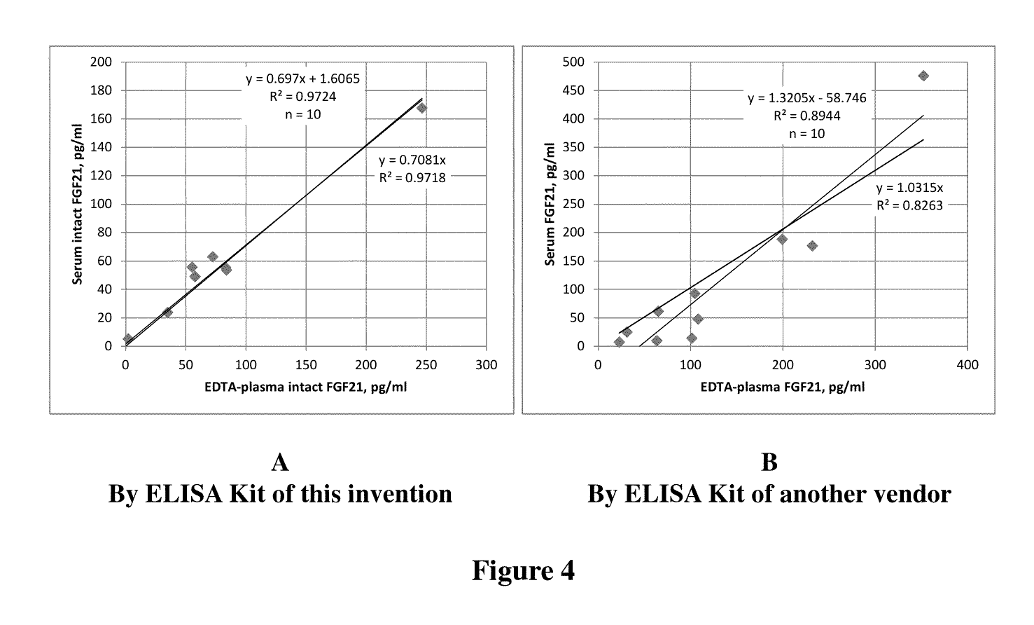

One method for evaluating the performance of an ELISA kit is to test the correlation of measurements in paired samples from the same donor. Levels of intact FGF21 in paired donor EDTA-plasma and serum samples were measured using the human intact FGF21 ELISA kit of the invention (FIG. 4A). As a comparison, levels of FGF21 in paired donor EDTA-plasma and serum samples were measured using an FGF21 ELISA kit of another vendor and the results were shown in FIG. 4B. The R² values of the linear regression of the paired measurements for the ELISA kit of this invention and that of another vendor are 0.97 and 0.82, respectively. These results showed that the measurements of FGF21 in paired donor plasma and serum samples using the invented method are very well correlated with each other (R²=0.97).

### Example 4

### Comparison of Measurements of FGF21 in Normal and Patient Samples Using Different Immunoassay Methods

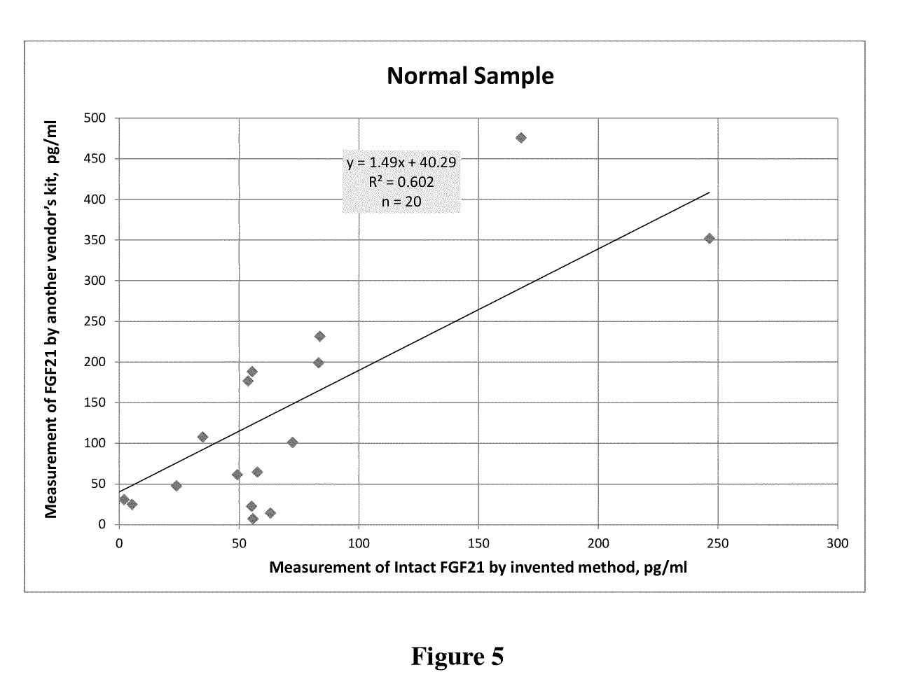

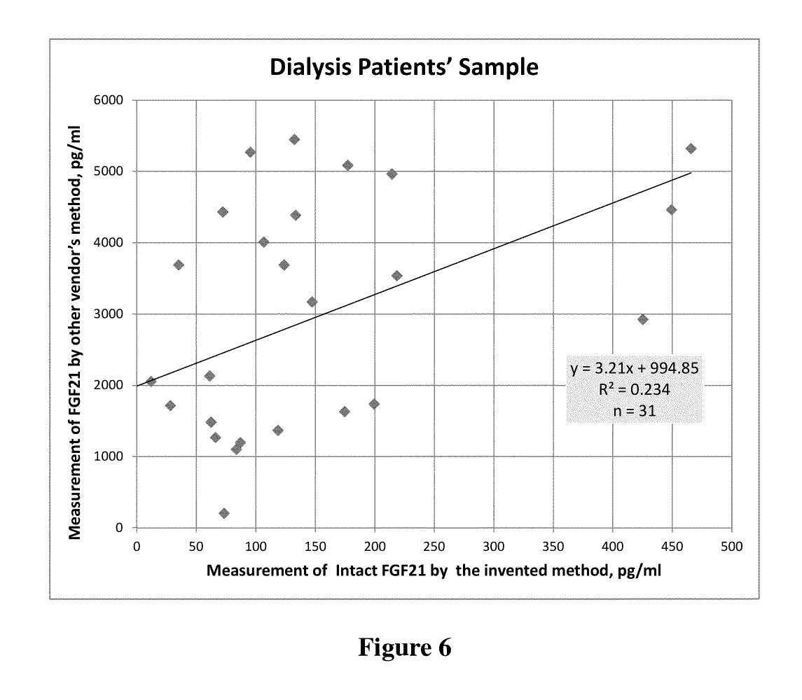

Levels of FGF21 in normal and Dialysis patients' samples were measured using the intact human FGF21 ELISA kit of this invention and an ELISA kit from another vendor (FIGS. 5 and 6). The intact human FGF21 ELISA kit specifically measures intact FGF21 in the samples. The other ELISA kit does not specify any epitope of FGF21 to be measured and seems to measure different forms of FGF21 in the samples. In both the normal and patients' samples, it is consistent that the measured values of FGF21 are always higher when using other ELISA kit than that of using the ELISA kit of the invention. The difference is especially big in dialysis patients' samples. The concentrations of intact FGF21 measured by the invented method ranged from 50 to 450 pg/ml while the concentrations of FGF21 and FGF21 fragments measured by other ELISA kit ranged from 1000 to 6000 pg/ml. The correlation of the two FGF21 measurements in the same patient sample is quite poor with a R² value of 0.23. These data suggest that FGF21 may be quickly digested into various fragments, each with different activities, in these patients. Measurement of mixed forms of FGF21 may not accurately reflect the biological activity of FGF21 in the sample, and could provide misleading information in clinical diagnosis.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

1. Yie J, et al. FGF21 N- and C-termini play different roles in receptor interaction and activation. FEBS Lett. 2009 Jan. 5; 583:19-24.
2. Micanovic R, et al. Different roles of N- and C-termini in the functional activity of FGF21. J Cell Physiol. 2009 May; 219(2):227-34.
3. Murata Y, et al. FGF21 as an Endocrine Regulator in Lipid Metabolism: From Molecular Evolution to Physiology and Pathophysiology. Journal of Nutrition and Metabolism, 2011, Article ID 981315, 8 pages.
4. Ptthoff M J, et al. Endocrine fibroblast growth factors 15/19 and 21: from feast to famine. Gene & Dev, 2012; 26: 312-24.
5. Hoogenboom HR. Selecting and screening recombinant antibody libraries. Nat. Biotechnol. 2005 September; 23(9):1105-16.
6. Antibodies: a laboratory manual. Eds. Harlow and Lane, Cold Spring Harbor Laboratory Press, NY, 1988
7. Bennett B, et al. A comparison of commercially available adjuvants for use in research. J Immunol Methods. 1992 Aug. 30; 153(1-2):31-40.

What is claimed is:

1. An isolated antibody, or an antigen-binding fragment thereof, specific for an N-terminal peptide sequence of fibroblast growth factor 21 (FGF21) wherein said N-terminal peptide sequence consists of HPIPDSSPLLQFGGQVRQ (SEQ ID NO:1) or HPIPDSS (SEQ ID NO:19), and wherein at least three amino acids in said N-terminal peptide sequence are part of a reactive portion with said antibody or antigen-binding fragment.

2. The antibody of claim 1, which is a monoclonal or a polyclonal antibody.

3. An isolated antibody, or antigen-binding fragment thereof, specific for a C-terminal peptide sequence of FGF21 wherein said C-terminal peptide sequence consists of DPLSMVGPSQGRSPSYAS (SEQ ID NO:2) or RSPSYAS (SEQ ID NO:20), and wherein at least three amino acids in said C-terminal peptide sequence are part of a reactive portion with said antibody or antigen-binding fragment.

4. The antibody of claim 3, which is a monoclonal or a polyclonal antibody.

5. A method for measuring an amount of intact FGF21 in a sample, comprising:
a) adding to said sample a first antibody or antigen-binding fragment specific for an N-terminal peptide sequence of FGF21;
b) adding to said sample a second antibody or antigen-binding fragment specific for a C-terminal peptide sequence of FGF21;
c) allowing said first and second antibody or antigen-binding fragment to bind to intact FGF21 in said sample, thereby forming a complex; and
d) measuring the amount of said complex to determine the amount of intact FGF21 in said sample while not detecting FGF21 fragments shorter than said intact FGF21.

6. The method of claim 5, wherein said N-terminal peptide sequence consists of HPIPDSSPLLQFGGQVRQ (SEQ ID NO:1), and wherein at least three amino acids in said N-terminal peptide sequence are part of a reactive portion with said first antibody or antigen-binding fragment.

7. The method of claim 5, wherein said C-terminal peptide sequence consists of DPLSMVGPSQGRSPSYAS (SEQ ID NO:2), and wherein at least three amino acids in said C-terminal peptide sequence are part of a reactive portion with said second antibody or antigen-binding fragment;

8. The method of claim 5, wherein said N-terminal peptide sequence is selected from the group consisting of SEQ ID NO:9, SEQ ID NO:10, SEQ ID NO:11, SEQ ID NO:12, SEQ ID NO:13 and SEQ ID NO:19, and wherein at least three amino acids in said N-terminal peptide sequence are part of a reactive portion with said first antibody or antigen-binding fragment.

9. The method of claim 5, wherein said C-terminal peptide sequence is selected from the group consisting of SEQ ID NO:14, SEQ ID NO:15, SEQ ID NO:16, SEQ ID NO:17, SEQ ID NO:18 and SEQ ID NO:20, and wherein at least three amino acids in said C-terminal peptide sequence are part of a reactive portion with said second antibody or antigen-binding fragment;

10. The method of claim 5, wherein one of said first and second antibody or antigen-binding fragment is a capture antibody, and the other of said first and second antibody or antigen-binding fragment is a detection antibody.

11. The method of claim 10, wherein said detection antibody fragment is fluorescently, chemically, radioactively or enzymatically labeled.

12. The method of claim 10, wherein said capture antibody is attached to a solid support.

13. The method of claim 5, wherein said complex is detected by a third labeled antibody that specifically binds to said first or second antibody.

14. A method for measuring an amount of FGF21 with an untruncated N-terminal in a sample, comprising:
a) adding to said sample an antibody or antigen-binding fragment specific for an N-terminal peptide sequence of FGF21 wherein at least three amino acids in said N-terminal peptide sequence are part of a reactive portion with said antibody or antigen-binding fragment;
b) allowing said antibody or antigen-binding fragment to bind to the N-terminal of FGF21 in said sample, thereby forming a complex; and
c) measuring the amount of said complex to measure the amount of FGF21 with an untruncated N-terminal in said sample while not detecting FGF21 fragments without an untruncated N-terminal.

15. The method of claim 14, wherein said N-terminal peptide sequence is selected from the group consisting of SEQ ID NO:1, SEQ ID NO:9, SEQ ID NO:10, SEQ ID NO:11, SEQ ID NO:12, SEQ ID NO:13 and SEQ ID NO:19.

16. A method for measuring an amount of FGF21 with an untruncated C-terminal in a sample, comprising:
a) adding to said sample an antibody or antigen-binding fragment specific for a C-terminal peptide sequence of FGF21 wherein at least three amino acids in said C-terminal peptide sequence are part of a reactive portion with said antibody or antigen-binding fragment;
b) allowing said antibody or antigen-binding fragment to bind to the C-terminal of FGF21 in said sample, thereby forming a complex; and
c) measuring the amount of said complex to measure the amount of FGF21 with an untruncated C-terminal in said sample while not detecting FGF21 fragments without an untruncated C-terminal.

17. The method of claim 16, wherein said C-terminal peptide sequence is selected from the group consisting of SEQ ID NO:2, SEQ ID NO:14, SEQ ID NO:15, SEQ ID NO:16, SEQ ID NO:17, SEQ ID NO:18 and SEQ ID NO:20.

18. A kit for measuring intact FGF21 in a sample, comprising:
a) an isolated antibody or antigen-binding fragment specific for an N-terminal peptide sequence of FGF21 consisting of HPIPDSSPLLQFGGQVRQ (SEQ ID NO:1) or HPIPDSS (SEQ ID NO:19), wherein at least three amino acids in said N-terminal peptide sequence are part of a reactive portion with said antibody or antigen-binding fragment; and
b) an isolated antibody or antigen-binding fragment specific for a C-terminal peptide sequence of FGF21 consisting of DPLSMVGPSQGRSPSYAS (SEQ ID NO:2) or RSPSYAS (SEQ ID NO:20), wherein at least three amino acids in said C-terminal peptide sequence are part of a reactive portion with said antibody or antigen-binding fragment.

19. A method for diagnosing a disease associated with increased or decreased levels of FGF21 in a person, comprising the steps of:
a) obtaining a sample from said person;
b) adding to said sample a first antibody or antigen-binding fragment specific for an N-terminal peptide sequence of FGF21 wherein said N-terminal peptide sequence consists of HPIPDSSPLLQFGGQVRQ (SEQ ID NO:1) or HPIPDSS (SEQ ID NO:19), and wherein at least three amino acids in said N-terminal peptide sequence are part of a reactive portion with said first antibody or antigen-binding fragment;
c) adding to said sample a second antibody or antigen-binding fragment specific for an C-terminal peptide sequence of FGF21 wherein said C-terminal peptide sequence consists of DPLSMVGPSQGRSPSYAS (SEQ ID NO:2) or RSPSYAS (SEQ ID NO:20), and wherein at least three amino acids in said c-terminal peptide sequence are part of a reactive portion with said second antibody or antigen-binding fragment;
d) allowing said first and second antibody or antigen-binding fragment to bind to intact FGF21 in said sample, thereby forming a complex;
e) measuring the amount of said complex to measure the amount of intact FGF21 in said sample while not detecting FGF21 fragments shorter than said intact FGF21; and
f) comparing the amount of intact FGF21 in said sample to the range of values from normal people to determine if the person has said disease.


SEQUENCE LISTING (20 sequences)

SEQ ID NO: 1 - protein, 18 aa, Homo sapiens
HPIPDSSPLLQFGGQVRQ

SEQ ID NO: 2 - protein, 18 aa, Homo sapiens
DPLSMVGPSQGRSPSYAS

SEQ ID NO: 3 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 4 - protein, 182 aa, Mus musculus (mouse)
AYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 5 - protein, 179 aa, Rattus norvegicus (rat)
YPISDSSPLLQFGGQVRQRYLYTDDDQDTEAHLEIREDGTVVGTAHRSPESLLELKALKPGVIQILGVKASRFLCQQPDGTLYGSPHFDPEACSFRELLLKDGYNVYQSEAHGLPLRLPQKDSQDPATRGPVRFLPMPGLPHEPQEQPGVLPPEPPDVGSSDPLSMVEPLQGRSPSYAS

SEQ ID NO: 6 - protein, 160 aa, Macaca mulatta (Rhesus Monkey)
HPIPDSSPLLQFGGQVRQRYLYTDDAQQTEAHLEIREDGTVGGAAHQSPESECGPEPGSEGGGALHFDPEACSFRELLLENGYNVYQSEAHGLPLHLPGNKSPHRDPASQGPARFLPLPGLPPAPPEPPGILAPQPPDVGSSDPLSMVGPSQARSPSYAS

SEQ ID NO: 7 - protein, 181 aa, Bos taurus (cattle)
HPIPDSSPLLQFGGQVRQRYLYTDDAQETEAHLEIRADGTVVGAARQSPESLLELKALKPGVIQILGVKTSRFLCQGPDGKLYGSLHFDPKACSFRELLLEDGYNVYQSETLGLPLRLPPQRSSNRDPAPRGPARFLPLPGLPAAPPDPPGILAPEPPDVGSSDPLSMVGPSYGRSPSYTS

SEQ ID NO: 8 - protein, 181 aa, Sus scrofa (pig)
RPIPDSSPLLQFGGQVRQRYLYTDDAQETEAHLEIRADGTVAGVARQSPESLLELKALKPGVIQILGVQTSRFLCQGPDGRLYGSLHFDPEACSFRELLLEDGYNVYQSEALGLPLRLPPHRSSNRDLAPRGPARFLPLPGLPPAPPEPPGILAPEPPDVGSSDPLSMVGPSHGRSPSYTS

SEQ ID NO: 9 - protein, 18 aa, Mus musculus (mouse)
AYPIPDSSPLLQFGGQVR

SEQ ID NO: 10 - protein, 18 aa, Rattus norvegicus (rat)
YPISDSSPLLQFGGQVRQ

SEQ ID NO: 11 - protein, 18 aa, Macaca mulatta (monkey)
HPIPDSSPLLQFGGQVRQ

SEQ ID NO: 12 - protein, 18 aa, Bos taurus (cattle)
HPIPDSSPLLQFGGQVRQ

SEQ ID NO: 13 - protein, 18 aa, Sus scrofa (pig)
RPIPDSSPLLQFGGQVRQ

SEQ ID NO: 14 - protein, 18 aa, Mus musculus (mouse)
DPLSMVEPLQGRSPSYAS

SEQ ID NO: 15 - protein, 18 aa, Rattus norvegicus (Rat)
DPLSMVEPLQGRSPSYAS

SEQ ID NO: 16 - protein, 18 aa, Macaca mulatta (monkey)
DPLSMVGPSQARSPSYAS

SEQ ID NO: 17 - protein, 17 aa, Bos taurus (cattle)
PLSMVGPSYGRSPSYTS

SEQ ID NO: 18 - protein, 18 aa, Sus scrofa (pig)
DPLSMVGPSHGRSPSYTS

SEQ ID NO: 19 - protein, 7 aa, Homo sapiens
HPIPDSS

SEQ ID NO: 20 - protein, 7 aa, Homo sapiens
RSPSYAS